(19) United States
(12) Patent Application Publication
(10) Pub. No.: US 2013/0030046 A1
(43) Pub. Date: Jan. 31, 2013

(54) USE OF BLACK SOYBEAN HULL EXTRACTS IN MANUFACTURE OF PRODUCTS FOR PREVENTING AND TREATING OSTEOARTHRITIS

(71) Applicants: Chunhua Li, Beijing (CN); Yanmei Li, Beijing (CN); Qinghua Liu, Beijing (CN)

(72) Inventors: Chunhua Li, Beijing (CN); Yanmei Li, Beijing (CN); Qinghua Liu, Beijing (CN)

(73) Assignee: BEIJING GINGKO GROUP BIOLOGICAL TECHNOLOGY CO., LTD., HAIDIAN DISTRICT, BEIJING (CN)

(21) Appl. No.: 13/576,748

(22) PCT Filed: Nov. 11, 2010
(86) PCT No.: PCT/CN10/78627

§ 371 Date: Oct. 16, 2012

(30) Foreign Application Priority Data
Feb. 2, 2010 (CN) 20101011327.8

(51) Int. Cl. A61K 31/352; A61P 19/02

(52) U.S. Cl. 514/456

(57) ABSTRACT

The application relates to a use of black soybean hull extracts in the manufacture of foods, health care products and medicaments. The black soybean hull extracts can be added to foods or drinks, and also can be made into all kinds of oral health care products and oral medicaments used by conventional methods of preparing traditional Chinese medicine preparations. The products can significantly improve symptoms of osteoarthritis, and also can stabilize or even reverse the degradation of cartilage of osteoarthritis.

### TECHNICAL FIELD

The application relates to the use of extracts originated from foods, and in particular, to the use of black soybean hull extracts in manufacture of foods, health care products and medicaments for preventing and treating osteoarthritis (OA).

### BACKGROUND

Black soybean, the Latin name for Glycine max (L.) Merr., is also referred to as black bean, soybean, horse bean or winter bean. According to the theory of traditional Chinese medicine, black materials can have an effect on kidney, the black soybean is sweet-warm, non-toxic, good for the kidney, the spleen and the heart, and has the effects of kidney tonifying and body building, dehumidification and damp clearing, as well as anti-aging. More and more researchers have found that the black soybean is of great nutrition, especially the study of pharmacological efficacy of black soybean hull has developed even further. The black soybean hull is black, also called soybean coat, soybean skin, black soybean coat, black soybean skin, bean coat, bean skin and so on.

The black soybean hull extracts contain anthocyanins, polyphenols and other active ingredients. The anthocyanins are not only a colorant, but also a functional component with the effect of health care in the black soybean. It not only improves the capillary circulation, but also has obvious anti-fatigue effect. The polyphenolys are important compounds in nature, which are an antioxidant.

The black soybean hull extracts improve eyesight, stop bleeding, are diuretic and antiscorbutic, protect capillary vessel, promote the regeneration of erythrocytes of eyes, enhance the ability to adapt to the darkness, can obviously improve asthenopia, and have obvious releasing effect on asthenopia suffered by teenagers, including blurred vision, bloated eyeball, ophthalmalgia, photophobia, xerotic eye and eyestrain. Furthermore, the black soybean hull extracts also have the effect of anti-oxidation and anti-ageing as well as free radical scavenging, and can be applied to people with hypertension, hyperlipemia and hyperglycemia to improve the quality of life. Japanese scholars report that the black soybean hull extracts also have good whitening effect, the mechanism of which is that the product having clear action and obvious effect is a good anti-oxidant and anti-ageing product by inhibiting tyrosine kinase.

### SUMMARY OF THE INVENTION

With the development of research and explore of the health care functions and pharmacological effects of the black soybean hull extracts, so far, there isn't any report about the use of black soybean hull extracts in preventing and treating osteoarthritis(OA). The inventor, when researching the black soybean hull extracts, discovered by accident that the black soybean hull extracts had obvious effect of inhibiting OA, and had a new use of preventing and treating OA.

OA is also known as degenerative osteoarthritis, which is degenerative in joint cartilage and forms bone spurs at the joint margins. OA manifests mostly stiff hands and feet, pain, swelling and friction sound, which is degenerative in joint cartilage and forms bone spurs at the joint margins. OA often offends old people, with the increasing longevity of our population, the incidence rate of OA is high, which seriously interferes with work. OA is the second reason why people above 50 lose their labour force, rank only second to heart disease. The incidence of OA in Western countries is especially high, according to statistics, accounting for 2.3% of outpatient cases. Pathological changes include inflammation hyperplasia adhesion of knee joint synovium, damage and destruction of joint cartilage and degenerative changes of perpatellar soft tissue and ligament. The principal pathogenesis mechanism includes endocrine change, weight gain, blood insufficiency around joint due to increased stress on knee joint, and bad neurotrophy. OA can be divided into primary one and secondary one. The cause for primary OA is not found, secondary osteoarthritis is developed from original diseases. OA is the common pathway for many diseases, including congenital abnormal joint development, joint lesion in childhood, trauma, various metabolic diseases and many inflammations in joints causing breakdown of cartilage.

Medicaments for treating OA clinically include painkillers, such as acetaminophen (i.e., Paracetamol); non-steroidal anti-inflammatory drugs such as adrenocortical hormones, Diprospan and Limethason locally injected in the joint cavity or lesion site; diclofenac sodium (including Votalin, Difene, Antine, diclofenac, Arthrotec), Sulindac, Rantudil, Neptunlong, Celebrex and Vioxx. Although some medicaments relieve the symptoms quickly, there may be serious side effects, mainly including gastrointestinal, cardiovascular and renal complications, without reversing arthritis. Some medicaments even aggravate the damage on joint cartilage.

Therefore, one of the goals of the application relates to the use of black soybean hull extracts in manufacture of health care products for preventing OA.

Another goal of the application relates to the use of black soybean hull extracts in manufacture of medicaments for treating OA.

Another goal of the application relates to the use of black soybean hull extracts in manufacture of foods for treating OA.

One aspect of the application is to provide health care products, which contain black soybean hull extracts, for preventing OA.

One aspect of the application is to provide medicaments, which contain black soybean hull extracts, for preventing OA containing.

One aspect of the application is to provide foods, which contain black soybean hull extracts, for preventing OA.

In one aspect of the application, the foods, medicaments and health care products consumed on a daily basis contain 100 mg to 800 mg of black soybean hull extracts for the purpose of preventing OA.

The above foods can be made into plain foods by a common method for making food, and the black soybean hull extracts are added.

The health care products and medicament can be prepared into various dosage forms needed by conventional methods of preparing traditional Chinese medicine preparations, including at least one of powders, decoction, tablets, capsules, granules and pills.

100 mg to 800 mg of black soybean hull extracts involved in the application are consumed on a daily basis as measured according to active ingredients, one course of treatment for 4 weeks, taken 3 courses of treatment continuously, and used as appropriate according to the individual situations and therapeutic purposes. The daily dosage can be 100 mg to 200 mg, 200 mg to 300 mg, 300 mg to 400 mg, 400 mg to 500 mg, 500 mg to 600 mg, 600 mg to 700 mg, 700 mg to 800 mg, more preferably 400 mg to 800 mg.

The black soybean hull extracts contain polyphenols. In the application, there aren't any special requirements for the content of polyphenols and extraction method. Due to different extraction methods, the content of the extracts might be varied to some extent. In phenolic compound of black soybean hull extracts, the content of total polyphenol is generally 50 weight % to 95 weight %, and the content of anthocyanin is 5 weight % to 30 weight. %.

There are many kinds of OA, the reason is also complex. The application has health care effects on all kinds of OA, in particular suitable for bone injury induced by degenerative osteoarthritis or wear due to joint ageing. In an embodiment of the application, patients with mild and moderate arthritis take the black soybean hull extracts. The severity of OA is evaluated by VAS score index and Lequesne index, by comparing the pain index before and after taking the black soybean hull extracts, it is found that the black soybean hull extracts obviously improve pain symptoms of patients suffering from OA. Safety studies are performed on blood pressure and heart rate of patients with OA, who have taken the black soybean hull extracts, as compared to the case before taking the black soybean hull extracts, no change shows. It proves that the black soybean hull extracts have safe and reliable effects on patients with OA, and are trustworthy foods, health care products and medicaments.

There are many kinds of health care products, which may be various oral products containing the black soybean hull extracts, including drinks and foods.

The application also provides a novel medicament for treating OA, which contains the black soybean hull extracts. The polyphenols as the active ingredients contained in the black soybean hull extracts play a role in OA active ingredients.

The application also provides a pharmaceutical composition containing the black soybean hull extracts. The active ingredients of the pharmaceutical composition are polyphenols contained in the black soybean hull extracts, and the pharmaceutical composition can be prepared by adding excipients.

In the above pharmaceutical composition, the content of total polyphenol is 50 weight % to 95 weight %, and the content of anthocyanin is 5 weight % to 30 weight %.

The above pharmaceutical composition contains 100 mg to 800 mg of black soybean hull extracts, the dosage of which can be 100 mg to 200 mg, 200 mg to 300 mg, 300 mg to 400 mg, 400 mg to 500 mg, 500 mg to 600 mg, 600 mg to 700 mg, 700 mg to 800 mg, more preferably 400 mg to 800 mg.

The above pharmaceutical composition can be prepared into various dosage forms clinically needed by conventional methods of preparing traditional Chinese medicine preparations, including at least one of powders, decoction, tablets, capsules, granules and pills.

100 mg to 800 mg of black soybean hull extracts of the application are consumed on a daily basis as measured according to active ingredients, one course of treatment for 4 weeks, taken 3 courses of treatment continuously, and used as appropriate according to the individual situations and therapeutic purposes.

The application provides a new use of black soybean hull extracts. The health care products, medicaments and foods containing the black soybean hull extracts can be used for preventing and treating primary and secondary osteoarthritis. The active ingredients not only can be prepared into pharmaceutical preparations, but also can be added to drinks and foods, thereby give full play to their role in prevention and in maintenance of health more conveniently. The application has the advantages of convenient use, no side effect, capability of significantly improving the pain symptoms of OA, thereby substantially improving the conditions, slowing down, stabilizing and reversing the degeneration of cartilage of OA.

### BRIEF DESCRIPTION OF THE DRAWINGS

FIG. 1 shows the effect of treating OA by taking 800 mg of black soybean hull extracts on a daily basis which is evaluated by VAS score index in the application.

FIG. 2 shows the effect of treating OA by taking 800 mg of black soybean hull extracts on a daily basis which is evaluated by Lequesne index in the application.

FIG. 3 shows the effect of treating OA by taking 400 mg of black soybean hull extracts on a daily basis which is evaluated by VAS score index in the application.

FIG. 4 shows the effect of treating OA by taking 400 mg of black soybean hull extracts on a daily basis which is evaluated by Lequesne index in the application.

FIG. 5 shows the effect of treating OA by taking 100 mg of black soybean hull extracts on a daily basis which is evaluated by VAS score index in the application.

FIG. 6 shows the effect of treating OA by taking 100 mg of black soybean hull extracts on a daily basis which is evaluated by Lequesne index in the application.

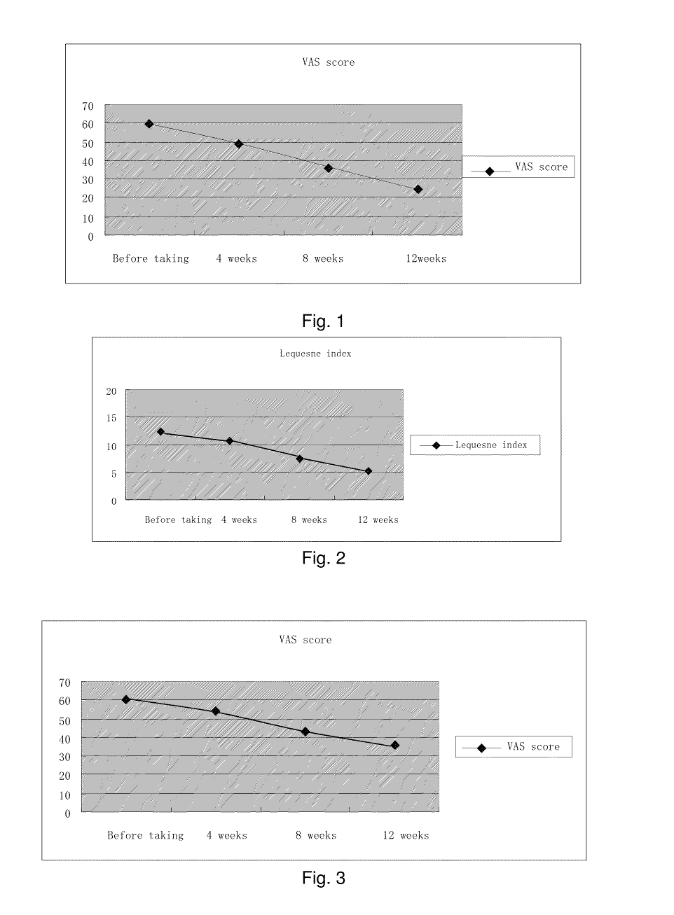

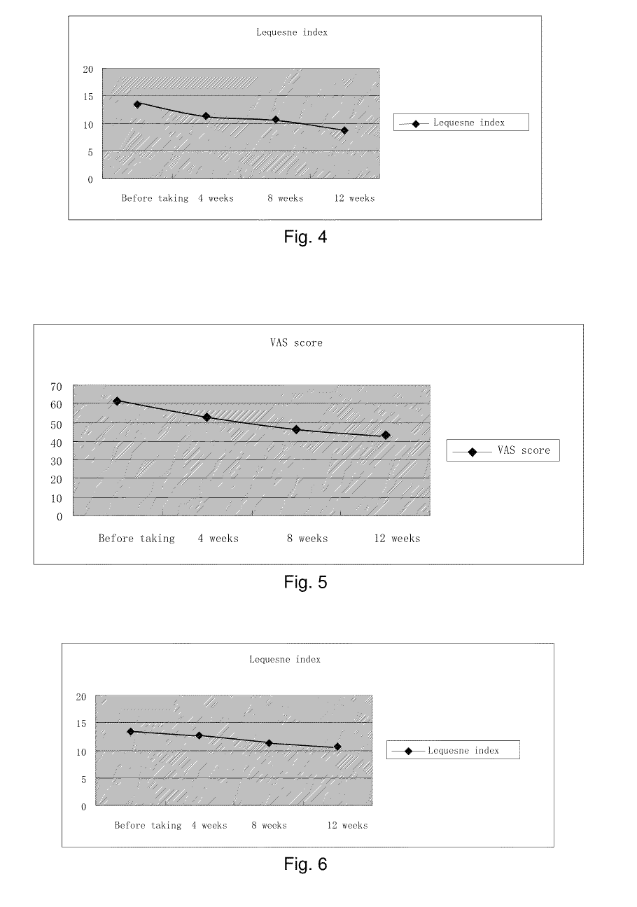

### DETAILED DESCRIPTION

Detailed description to the pharmaceutical composition for treating OA of the application is shown as follows, clinical trial examples and preparation examples.

### I. Clinical Trial Example 1

In order to understand the essence of the application better, the new use of black soybean hull extracts in preparing medicaments for treating OA is illustrated better by the following clinical trials.

The Western Ontario and McMaster Universities (WOMAC) OA index [0˜100 mm Visual Analogue Scale (VAS)] and Lequesne pain function index results are used for analysis.

WOMAC score is more widely used in OA literatures. From the content, this score evaluates the structure and function of knee joint in terms of pain, stiffness and joint function, covering fundamental symptoms and physical signs of the overall OA. WOMAC score is effective in that some situations about the patients before and after treatment can be reflected accurately, such as satisfaction degree of patients on treatment. In relative terms, this score has higher reliability for the evaluation of OA.

Lequesne index is an international scoring standard commonly used for OA, first put forward by Lequesne MG in 1991, used for evaluating the severity of OA of the hip and the knee. The scoring standard is widely used in Europe, particularly beneficial for long-term effect indicator of drug therapy. Evaluation item includes night pain, motion pain, morning stiffness or gain after getting up, distance, and daily activities (e.g., going up the stairs, going down the stairs, squats, etc.), each also includes a plurality of small parts, each having different point values. Finally, the functional status of patients with knee OA is evaluated according to total scores (the higher the index, the more serious are the symptoms), which is relatively objective and comprehensive.

### 1. Case Selection:

All of the cases were randomly selected from patients with knee joint OA of orthopedic clinic from January to September in 2008, a total of 10 cases, including 3 males and 7 females, aging from 41 to 78 years old with average of 55 years old. The shortest course of disease was 1 year, the longest was 12 years, and the average was (6.3±2.8) years. All of the cases accorded with the diagnostic criteria for knee joint OA formulated by American Rheumatism Association. The X-ray classification of knee joint OA is Class 1, Class 2 and Class 3 according to Kellgren standard. All the selected cases had not received any drug treatment or stop taking the drugs over 2 weeks, and excluded the patients with active gastrointestinal diseases, kidney diseases, liver diseases or blood coagulation disorders, the patients diagnosed with inflammatory arthritis, gout or acute knee arthritis trauma and the patients allergic to NSAID or sulfonamide.

### 2. Grouping:

By a self control method, the black soybean hull extracts were taken (the content of total polyphenol was 95% and the content of anthocyanin was 30%), 400 mg each time, once in the morning and once in the evening, one course of treatment for 4 weeks, taken 3 courses of treatment orally.

### 3. Efficacy Evaluation

All of the cases had taken the drug and been followed up for one year. The joint function was measured respectively before and after taking the drug for 4 weeks, 8 weeks and 12 weeks. The WOMAC OA index [0˜100 mm Visual Analogue Scale (VAS)] and Lequesne pain function index results were used for analysis.

### 4. Statistical Processing

All data was represented by mean±standard deviation, which was for t test, there were significantly different at P<0.05.

### 5. Experimental Result

The joint pain symptoms suffered by the patients with OA were significantly reduced by taking the black soybean hull extracts as compared to the case before treatment, both the VAS score and the Lequesne index were reduced, the values measured showed P<0.05 when t test was performed, and there were significantly different. It showed that the black soybean hull extracts were effective to treat knee OA. The details can be observed in Table 3.

TABLE 1
Influence of black soybean hull extracts on VAS score index
changes of OA severity ( x̅ ± s, n = 15)

| Patient | Before | | | |
|---|---|---|---|---|
| No. | taking | 4 weeks | 8 weeks | 12 weeks |
| 1 | 59.5 | 50.4 | 37.8 | 24.3 |
| 2 | 61.5 | 51.6 | 39.3 | 25.4 |
| 3 | 60.6 | 49.8 | 37.5 | 24.6 |
| 4 | 62.5 | 51.7 | 39.2 | 26.4 |
| 5 | 58.6 | 49.3 | 38.1 | 25.4 |
| 6 | 58.8 | 49.6 | 40.3 | 27.1 |
| 7 | 61.3 | 50.7 | 39.1 | 26.4 |
| 8 | 60.3 | 50.5 | 37.5 | 23.8 |
| 9 | 60.8 | 51.3 | 37.2 | 24.2 |
| 10 | 59.3 | 49.5 | 38.2 | 25.6 |
| VAS | 60.32 ± 1.24 | 50.44 ± 1.02¹⁾ | 38.42 ± 1.00¹⁾ | 25.32 ± 1.03¹⁾ |
| score | | | | |
| index | | | | |

Compared with that before taking: ¹⁾P < 0.05

TABLE 2
Influence of black soybean hull extracts on Lequesne index changes of
OA severity ( x̅ ± s, n = 15)

| | Before | | | |
|---|---|---|---|---|
| Patient No. | taking | 4 weeks | 8 weeks | 12 weeks |
| 1 | 13.8 | 11.3 | 8.7 | 6.5 |
| 2 | 14.3 | 11.8 | 9.1 | 6.9 |
| 3 | 13.3 | 10.7 | 8.1 | 6.2 |
| 4 | 15.2 | 12.7 | 10.1 | 8.0 |
| 5 | 13.8 | 11.4 | 8.8 | 6.8 |
| 6 | 13.9 | 11.5 | 8.7 | 6.5 |
| 7 | 14.2 | 11.7 | 8.9 | 6.9 |
| 8 | 13.1 | 10.6 | 7.9 | 6.5 |
| 9 | 13.2 | 11.8 | 9.1 | 6.9 |
| 10 | 12.9 | 10.5 | 7.7 | 6.5 |
| Lequesne | 13.77 ± 0.62 | 11.40 ± 0.64¹⁾ | 8.71 ± 0.64¹⁾ | 6.77 ± 0.50¹⁾ |
| index | | | | |

Compared with that before taking: ¹⁾P < 0.05

TABLE 3
Influence of black soybean hull extracts on index changes of OA severity
( x̅ ± s, n = 15)

| | | Before | | | |
|---|---|---|---|---|---|
| Group | n | taking | 4 weeks | 8 weeks | 12 weeks |
| VAS score | 10 | 60.32 ± 1.24 | 50.44 ± 1.02¹⁾ | 38.42 ± 1.00¹⁾ | 25.32 ± 1.03¹⁾ |
| Lequesne | 10 | 13.77 ± 0.62 | 11.40 ± 0.64¹⁾ | 8.71 ± 0.64¹⁾ | 6.77 ± 0.50¹⁾ |
| index | | | | | |

Compared with that before taking: ¹⁾P < 0.05

### 6. Safety Result

TABLE 4
Blood pressure changes (/mmHg) and heart rate changes (times/min) of
patients with OA before and after taking black soybean hull extracts
( x̅ ± s, n = 15)

| Group | Before taking | 4 weeks | 8 weeks | 12 weeks |
|---|---|---|---|---|
| Low pressure | 86.45 ± 4.52 | 86.55 ± 4.44 | 86.89 ± 4.92 | 86.54 ± 4.82 |
| High pressure | 134.32 ± 11.26 | 134.45 ± 10.78 | 134.43 ± 10.45 | 134.65 ± 10.64 |
| Heart rate | 75 ± 1 | 75 ± 2 | 74 ± 3 | 75 ± 2 |

In t test, P > 0.05, there was no significant difference as compared to that before taking black soybean hull extracts.

A total of 10 middle and old aged patients with knee OA were recruited in the study. By an open clinical observation test method, the efficacy and safety of black soybean hull extracts for treating the patients with OA were studied. The result showed that 800 mg of black soybean hull extracts were taken every day, and continuously taken for 12 weeks, with a better curative effect achieved, the patients gave a good comprehensive evaluation on the curative effect. It should be noted that, the patients exhibited serious arthritis symptoms, with moderate and severe pain when recruited. After the patients took the drugs, the VAS score for the overall pain decreases progressively, and no pain VAS rebound occurred after the drug was taken; the Lequesne index also decreases progressively, and no rebound phenomena occurred. It prompted that the black soybean hull extracts have long duration and good effect of relieving pain.

### II. Clinical Trial Example 2

### 1. Case Selection:

All of the cases were randomly selected from patients with knee joint OA of orthopedic clinic from January to September in 2008, a total of 10 cases, including 1 male and 9 females, aging from 41 to 78 years old with average of 55 years old. The shortest course of disease was 1 year, the longest was 12 years, and the average was (6.3±2.8) years. All of the cases accorded with the diagnostic criteria for knee joint OA formulated by American Rheumatism Association. The X-ray classification of knee joint OA is Class 1, Class 2 and Class 3 according to Kellgren standard. All the selected cases had not received any drug treatment or stop taking the drugs over 2 weeks, and excluded the patients with active gastrointestinal diseases, kidney diseases, liver diseases or blood coagulation disorders, the patients diagnosed with inflammatory arthritis, gout or acute knee arthritis trauma and the patients allergic to NSAID or sulfonamide.

### 2. Grouping:

By a self control method, the black soybean hull extracts were taken (the content of total polyphenol was 75% and the content of anthocyanin was 15%), 200 mg each time, once in the morning and once in the evening, one course of treatment for 4 weeks, taken 3 courses of treatment orally.

### 3. Efficacy Evaluation

All of the cases had taken the drug and been followed up for one year. The joint function was measured respectively before and after taking the drug for 4 weeks, 8 weeks and 12 weeks. The WOMAC OA index [0˜100 mm Visual Analogue Scale (VAS)] and Lequesne pain function index results were used for analysis.

### 4. Statistical Processing

All data was represented by mean±standard deviation, which was for t test, there were significantly different at P<0.05.

### 5. Experimental Result

The joint pain symptoms suffered by the patients with OA were significantly reduced by taking the black soybean hull extracts as compared to the case before treatment, both the VAS score and the Lequesne index were reduced, the values measured showed P<0.05 when t test was performed, and there were significantly different. It showed that the black soybean hull extracts were effective to treat knee OA. The details can be observed in Table 7.

TABLE 5
Influence of black soybean hull extracts on VAS score index
changes of OA severity ( x̅ ± s, n = 10)

| Patient | Before | | | |
|---|---|---|---|---|
| No. | taking | 4 weeks | 8 weeks | 12 weeks |
| 1 | 59.4 | 52.4 | 43.7 | 34.3 |
| 2 | 61.6 | 54.3 | 45.6 | 35.9 |
| 3 | 60.8 | 53.2 | 44.3 | 34.7 |
| 4 | 62.5 | 55.1 | 45.4 | 35.8 |
| 5 | 59.6 | 54.4 | 45.3 | 35.7 |
| 6 | 59.5 | 52.5 | 44.2 | 34.6 |
| 7 | 60.8 | 53.1 | 44.3 | 34.8 |
| 8 | 61.3 | 54.1 | 45.2 | 35.7 |
| 9 | 61.4 | 55.2 | 46.1 | 35.4 |
| 10 | 60.4 | 53.5 | 44.6 | 34.9 |
| VAS score | 60.73 ± 1.02 | 53.78 ± 1.0¹⁾ | 38.67 ± 1.00¹⁾ | 25.47 ± 1.03¹⁾ |

Compared with that before taking: ¹⁾P < 0.05

TABLE 6
Influence of black soybean hull extracts on Lequesne index changes of
OA severity ( x̅ ± s, n = 10)

| Patient | Before | | | |
|---|---|---|---|---|
| No. | taking | 4 weeks | 8 weeks | 12 weeks |
| 1 | 14.1 | 12.4 | 10.6 | 9.3 |
| 2 | 13.8 | 12.2 | 10.7 | 9.4 |
| 3 | 14.2 | 12.4 | 10.9 | 8.9 |
| 4 | 14.3 | 12.7 | 11.1 | 9.3 |
| 5 | 13.7 | 12.1 | 10.8 | 9.5 |
| 6 | 14 | 12.3 | 10.5 | 9.2 |
| 7 | 14.2 | 12.5 | 11.2 | 9.4 |
| 8 | 14.4 | 12.8 | 11.5 | 9.8 |
| 9 | 14.3 | 12.6 | 10.2 | 9.1 |
| 10 | 13.9 | 12.3 | 10.4 | 9.2 |
| Lequesne | 14.11 ± 0.23 | 12.44 ± 0.22¹⁾ | 10.83 ± 0.40¹⁾ | 9.32 ± 0.24¹⁾ |
| index | | | | |

Compared with that before taking: ¹⁾P < 0.05

TABLE 7
Influence of black soybean hull extracts on index changes of OA severity
( x̅ ± s, n = 10)

| | | Before | | | |
|---|---|---|---|---|---|
| Group | n | taking | 4 weeks | 8 weeks | 12 weeks |
| VAS score | 10 | 60.73 ± 1.02 | 53.78 ± 1.0¹⁾ | 44.87 ± 0.75¹⁾ | 35.18 ± 0.58¹⁾ |
| Lequesne | 10 | 14.11 ± 0.23 | 12.44 ± 0.22¹⁾ | 10.83 ± 0.40¹⁾ | 9.32 ± 0.24¹⁾ |
| index | | | | | |

Compared with that before taking: ¹⁾P < 0.05

### 6. Safety Result

TABLE 8
Blood pressure changes (/mmHg) and heart rate changes (times/min) of
patients with OA before and after taking black soybean hull extracts
( x̅ ± s, n = 10)

| Group | Before taking | 4 weeks | 8 weeks | 12 weeks |
|---|---|---|---|---|
| Low pressure | 80.32 ± 3.32 | 80.12 ± 2.23 | 80.34 ± 2.12 | 80.56 ± 3.12 |
| High pressure | 130.23 ± 10.12 | 130.45 ± 10.28 | 130.34 ± 10.23 | 130.32 ± 11.21 |
| Heart rate | 78 ± 1 | 78 ± 2 | 77 ± 3 | 77 ± 2 |

In t test, P > 0.005, there was no significant difference as compared to that before taking black soybean hull extracts.

A total of 10 middle and old aged patients with knee OA were recruited in the study. By an open clinical observation test method, the efficacy and safety of black soybean hull extracts for treating the patients with OA are studied. The result showed that 400 mg of black soybean hull extracts were taken every day, and continuously taken for 12 weeks, with a better curative effect achieved, the patients gave a good comprehensive evaluation on the curative effect. It should be noted that, the patients exhibited serious arthritis symptoms, with moderate and severe pain when recruited. After the patients took the drugs, the VAS score for the overall pain decreases progressively, and no pain VAS rebound occurred after the drug was taken; the Lequesne index also decreases progressively, and no rebound phenomena occurred. It prompted that the black soybean hull extracts have long duration and good effect of relieving pain.

### III. Clinical Trial Example 3

### 1. Case Selection:

All of the cases were randomly selected from patients with knee joint OA of orthopedic clinic from January to September in 2008, a total of 10 cases, including 2 males and 8 females, aging from 41 to 78 years old with average of 55 years old. The shortest course of disease was 1 year, the longest was 12 years, and the average was (6.3±2.8) years. All of the cases accorded with the diagnostic criteria for knee joint OA formulated by American Rheumatism Association. The X-ray classification of knee joint OA is Class 1, Class 2 and Class 3 according to Kellgren standard. All the selected cases had not received any drug treatment or stop taking the drugs over 2 weeks, and excluded the patients with active gastrointestinal diseases, kidney diseases, liver diseases or blood coagulation disorders, the patients diagnosed with inflammatory arthritis, gout or acute knee arthritis trauma and the patients allergic to NSAID or sulfonamide.

### 2. Grouping:

By a self control method, the black soybean hull extracts were taken (the content of total polyphenol was 50% and the content of anthocyanin was 10%), 50 mg each time, once in the morning and once in the evening, one course of treatment for 4 weeks, taken 3 courses of treatment orally.

### 3. Efficacy Evaluation

All of the cases had taken the drug and been followed up for one year. The joint function was measured respectively before and after taking the drug for 4 weeks, 8 weeks and 12 weeks. The WOMAC OA index [0˜100 mm Visual Analogue Scale (VAS)] and Lequesne pain function index results were used for analysis.

### 4. Statistical Processing

All data was represented by mean±standard deviation, which was for t test, there were significantly different at P<0.05.

### 5. Experimental Result

The joint pain symptoms suffered by the patients with OA were significantly reduced by taking the black soybean hull extracts as compared to the case before treatment, both the VAS score and the Lequesne index were reduced, the values measured showed P<0.05 when t test was performed, and there were significantly different. It showed that the black soybean hull extracts were effective to treat knee OA. The details can be observed in Table 11.

TABLE 9
Influence of black soybean hull extracts on VAS score index changes
of OA severity ( x̅ ± s, n = 10)

| Patient | Before | | | |
|---|---|---|---|---|
| No. | taking | 4 weeks | 8 weeks | 12 weeks |
| 1 | 60.2 | 55.7 | 49.7 | 40.1 |
| 2 | 60.3 | 55.8 | 49.6 | 40.3 |
| 3 | 61.1 | 55.9 | 49.3 | 40.5 |
| 4 | 61.3 | 55.6 | 49.9 | 40.4 |
| 5 | 60.4 | 55.5 | 49.7 | 40.7 |
| 6 | 61.5 | 56.2 | 50.2 | 41.1 |
| 7 | 60.2 | 56.1 | 50.3 | 41.2 |
| 8 | 60.5 | 55.9 | 49.5 | 40.6 |
| 9 | 60.6 | 55.8 | 49.6 | 41.3 |
| 10 | 60.8 | 55.4 | 49.9 | 39.9 |
| VAS score | 60.69 ± 0.47 | 55.79 ± 0.26 | 49.77 ± 0.31¹⁾ | 40.61 ± 0.47¹⁾ |
| index | | | | |

Compared with that before taking: ¹⁾P < 0.05

TABLE 10
Influence of black soybean hull extracts on Lequesne index changes of
OA severity ( x̅ ± s, n = 10)

| Patient | Before | | | |
|---|---|---|---|---|
| No. | taking | 4 weeks | 8 weeks | 12 weeks |
| 1 | 13.8 | 12.8 | 11.2 | 10.2 |
| 2 | 13.9 | 12.9 | 11.4 | 10.5 |
| 3 | 14.1 | 13.1 | 12.1 | 10.9 |
| 4 | 13.5 | 12.5 | 12 | 10.8 |
| 5 | 13.6 | 12.4 | 11.5 | 10.4 |
| 6 | 14.2 | 13.6 | 11.8 | 10.7 |
| 7 | 14 | 13.1 | 11.6 | 10.8 |
| 8 | 14.4 | 12.6 | 11.7 | 10.6 |
| 9 | 14 | 12.9 | 11.3 | 10.9 |
| 10 | 13.9 | 12.3 | 11.5 | 10.3 |
| Lequesne [symbol] | 13.94 ± | 12.88 ± 0.39¹⁾ | 11.62 ± 0.29¹⁾ | 10.64 ± 0.25¹⁾ |
| | 0.27 | | | |

Compared with that before taking: ¹⁾P < 0.05

TABLE 11
Influence of black soybean hull extracts on index changes of OA
severity ( x̅ ± s, n = 10)

| | | Before | | | |
|---|---|---|---|---|---|
| Group | n | taking | 4 weeks | 8 weeks | 12 weeks |
| VAS | 10 | 60.69 ± 0.47 | 55.79 ± 0.26 | 49.77 ± 0.31¹⁾ | 40.61 ± 0.47¹⁾ |
| score | | | | | |
| Lequesne | 10 | 13.94 ± 0.27 | 12.88 ± 0.39¹⁾ | 11.62 ± 0.29¹⁾ | 10.64 ± 0.25¹⁾ |
| index | | | | | |

Compared with that before taking: ¹⁾P < 0.05

### 6. Safety Result

TABLE 12
Blood pressure changes (/mmHg) and heart rate changes (times/min)
of patients with OA before and after taking black soybean hull extracts
( x̅ ± s, n = 10)

| Group | Before taking | 4 weeks | 8 weeks | 12 weeks |
|---|---|---|---|---|
| Low pressure | 85.14 ± 3.34 | 85.32 ± 3.54 | 85.41 ± 2.34 | 85.34 ± 3.45 |
| High pressure | 134.12 ± 6.54 | 134.23 ± 7.23 | 134.15 ± 6.34 | 134.21 ± 7.56 |
| Heart rate | 75 ± 3 | 75 ± 2 | 74 ± 4 | 75 ± 1 |

In t test, P > 0.05, there was no significant difference as compared to that before taking black soybean hull extracts.

A total of 10 middle and old aged patients with knee OA were recruited in the study. By an open clinical observation test method, the efficacy and safety of black soybean hull extracts for treating the patients with OA were studied. The result showed that 100 mg of black soybean hull extracts were taken every day, and continuously taken for 12 weeks, with a better curative effect achieved, the patients gave a good comprehensive evaluation on the curative effect. It should be noted that, the patients exhibited serious arthritis symptoms, with moderate and severe pain when recruited. After the patients took the drugs, the VAS score for the overall pain decreases progressively, and no pain VAS rebound occurred after the drug was taken; the Lequesne index also decreases progressively, and no rebound phenomena occurred. It prompted that the black soybean hull extracts have long duration and good effect of relieving pain.

### II. Preparation Examples

A method for preparing products for treating OA which contains black soybean hull extracts was illustrated below through embodiments.

The black soybean hull extracts may be extracted by a conventional method in the art or commercially available. A method for preparing black soybean hull extracts of the application was referred to a method provided by Patent Application No. PCT/CN2006/003397, Publication No. US2008/0145482A1.

### Embodiment 1

### Preparation of Foods Containing Black Soybean Hull Extracts Making Fruit Salad

Step 1: 50 g of pineapples, 50 g of apples, 50 g of kiwis and 50 g of bananas were cut into dices like cube sugar, a plurality of cherry tomatoes were divided into four sectors; Step 2: 500 mg of black soybean hull extracts, a little sugar and one glass of red wine were added, stirred uniformly and marinated for 1 hour in a refrigerator; Step 3: the mixture was taken out of the refrigerator after 1 hour, then cherries without stalks were added to the mixture to obtain the fruit salad, which can be eaten once or several times.

### Embodiment 2

### Preparation of Drinks Containing Black Soybean Hull Extracts of the Application

6.2% of syrup with a concentration of 65%, 0.3% of citric acid with a concentration of 50%, 0.06% of sodium benzoate solution with a concentration of 25%, 0.16% of citrate sodium solution with a concentration of 25%, 0.01% of aspartame, 0.1% of jasmine essence, 15% of black soybean hull extract, an appropriate amount of caramel pigment were added, then water was added to 100%. Such drinks can be drank continuously for many times and days.

### Embodiment 3

### Preparation of Capsules of Medicament of the Application

200 mg of black soybean hull extracts were extracted by a conventional method in the art, which include 75% of total polyphenol and 15% of anthocyanin, smashed into powders to which 200 mg of starch was added, then the mixture was packed into gelatin capsules, 1 capsule daily.

### Embodiment 4

### Preparation of Tablets of Medicament of the Application

100 mg of black soybean hull extracts were extracted by a conventional method in the art, which include 50% of total polyphenol and 30% of anthocyanin, smashed into powders to which conventional auxiliary materials were added, then the mixture was formulated into tablets, 2 times per day.

### Embodiment 5

### Preparation of Granules of Medicament of the Application

150 mg of black soybean hull extracts were extracted by a conventional method in the art, which include 95% of total polyphenol and 5% of anthocyanin, smashed into powders to which conventional auxiliary materials were added, then the mixture was formulated into granules, once per day.

### Embodiment 6

### Preparation of Health Product Formula of the Application

The black soybean hull extracts, chondroitin sulfate, glucosamine, coenzyme Q10, and a certain amount of starch were added, mixed uniformly and prepared into capsules, with each capsule containing 200 mg of black soybean hull extracts, 40 mg of chondroitin sulfate, 30 mg of glucosamine and 15 mg of coenzyme Q10, 1 time to 2 times per day, 2 capsules each time, being used for treating OA.

Similarly, these products can be prepared into decoction, powders, pills or other dosage forms by the conventional method in the art. The amount of black soybean hull extracts in foods, health care products and medicaments of various dosage forms is 100 mg to 800 mg.

The above details the new use of black soybean hull extracts provided by the application in preventing and treating OA. It should be noted that the content described in the detailed description of the application is implementation mode preferred to implement the application better, the scope of protection of the application is not limited to the technical schemes described in the above implementation mode, shall be determined by the essential content of the claims, any possible changes in process shall fall within the scope of protection of the application without departing from the essential content of the claims.

1. A use of black soybean hull extracts in manufacture of products for preventing or treating osteoarthritis, the products comprising foods, health care products and medicaments.

2. The use of black soybean hull extracts according to claim 1, wherein the black soybean hull extracts contain polyphenols, the content of total polyphenol is 50 weight. % to 95 weight. %, the content of anthocyanin is 5 weight. % to 30 weight. %.

3. The use of black soybean hull extracts according to claim 1, wherein the osteoarthritis includes bone injury induced by degenerative osteoarthritis or wear due to joint ageing.

4. The use of black soybean hull extracts according to claim 1, wherein 100 mg to 800 mg of the black soybean hull extracts are contained in the products consumed on a daily basis.

5. The use of black soybean hull extracts according to claim 1, wherein the dosage form of the products containing the black soybean hull extracts is at least one selected from the group consisting of powders, oral liquids, tablets, capsules, granules and pills.

6. A medicament for treating osteoarthritis, wherein the medicament contains black soybean hull extracts.

7. The medicament according to claim 6, wherein the black soybean hull extracts contain polyphenols, the content of total polyphenol is 50 weight. % to 95 weight. %, the content of anthocyanin is 5 weight. % to 30 weight. %.

8. The medicament according to claim 7, wherein 100 mg to 800 mg of the black soybean hull extracts are contained in the medicament consumed on a daily basis.

9. The medicament according to claim 6, wherein the dosage form of the medicament is at least one selected from the group consisting of powders, oral liquids, tablets, capsules, granules and pills.

10. The medicament according to claim 6, wherein the osteoarthritis includes bone injury induced by degenerative osteoarthritis or wear due to joint ageing.

11. The medicament according to claim 7, wherein the dosage form of the medicament is at least one selected from the group consisting of powders, oral liquids, tablets, capsules, granules and pills.

12. The medicament according to claim 8, wherein the dosage form of the medicament is at least one selected from the group consisting of powders, oral liquids, tablets, capsules, granules and pills.
